(19) United States
(12) Patent Application Publication
(10) Pub. No.: US 2026/0177826 A1
(43) Pub. Date: Jun. 25, 2026

(54) ARRANGEMENT FOR FORMING AN IMAGE OF AN EYE OF A USER

(71) Applicant: Lumus Ltd., Ness Ziona (IL)

(72) Inventor: Eitan RONEN, Rechovot (IL)

(21) Appl. No.: 19/125,374

(22) PCT Filed: Oct. 24, 2023
(86) PCT No.: PCT/IL2023/051102

§ 371 Date: Apr. 29, 2025

Related U.S. Application Data
(60) Provisional application No. 63/444,957, filed on Feb. 12, 2023.

(51) Int. Cl. G02B 27/01; A61B 3/113; F21V 8/00

(52) U.S. Cl. CPC G02B 27/0172; A61B 3/113; G02B 6/0036; G02B 6/0038; G02B 6/0055; G02B 2027/0178

(57) ABSTRACT

An arrangement for forming an image of an eye of a user for tracking eye motion includes a lightguide (20) containing an obliquely-angled internal reflector (21), an image sensor (10) and a lens (11) associated with the lightguide for focusing light reflected from the eye of the user and reflected by the internal reflector onto the image sensor. A width of the internal reflector, an effective aperture of the lens and deployment of the image sensor are such that light from different areas of the eye and reflected by the internal reflector reaches the lens via one of at least three distinct paths, resulting in discrete non-overlapping image regions on the image sensor. The sub-images derived from these regions of the sensor can be manipulated and combined to form an image of the eye.

### FIELD AND BACKGROUND OF THE INVENTION

The present invention relates to eye tracking and, in particular, it concerns an arrangement for forming an image of an eye of a user via a lightguide in facing relation to the user's eye. The invention may be used to advantage in the context of a lightguide-based near-eye display.

Many near-eye display systems include a transparent lightguide or “lightguide” placed before the eye of the user, which conveys an image within the lightguide by internal reflection and then couples out the image by a suitable output coupling mechanism towards the eye of the user. The output coupling mechanism may be based on embedded partially-reflecting surfaces or “facets,” or may employ a diffractive pattern.

Some lightguide-based displays employ a lightguide arrangement which achieves expansion of an optical aperture of an image projector in two dimensions in order to employ a miniature projector to provide a much larger viewing area to the eye. Two-dimensional expansion can be achieved by employing an additional set of embedded partially-reflecting surfaces within the same lightguide, for example, as disclosed in PCT Patent Application Publication No. WO 2020/049542 A1, or by employing a separate rectangular lightguide, for example, as disclosed in PCT Patent Application Publication No. WO 2018/065975 A1, and may include various combinations of reflective and diffractive optical elements, as disclosed in PCT Patent Application Publication No. WO 2018/154576 A1.

It is desirable for many applications to track eye movements while a user is viewing a near-eye display. For this purpose, it is desirable to obtain an image of the eye via a lightguide placed in facing relation to the eye, which may be a lightguide of the display or an additional dedicated lightguide.

### SUMMARY OF THE INVENTION

The present invention is an arrangement for forming an image of an eye of a user via a lightguide in facing relation to the user's eye.

According to the teachings of an embodiment of the present invention there is provided, an arrangement for forming an image of an eye of a user for tracking eye motion, the arrangement comprising: (a) a lightguide formed from transparent material having a first major surface and a second major surface, the first and second major surfaces being planar and mutually parallel, the first major surface being deployed in facing relation to the eye of the user so that the user views a scene through the lightguide; (b) an obliquely-angled internal reflector deployed within the lightguide; (c) an image sensor comprising a two-dimension array of pixel sensors; and (d) a lens associated with the lightguide for focusing light reflected from the eye of the user and reflected by the internal reflector onto the image sensor, wherein a width of the internal reflector, an effective aperture of the lens and deployment of the image sensor are such that: (i) light from the eye of the user reflected by the internal reflector and reaching the lens without reflection from either the first or the second major surface is incident on a first region of the image sensor; (ii) light from the eye of the user reflected by the internal reflector and reaching the lens after a single reflection from the first major surface and without reflection from the second major surface is incident on a second region of the image sensor; and (iii) light from the eye of the user reflected by the internal reflector and reaching the lens after a single reflection from the second major surface and without reflection from the first major surface is incident on a third region of the image sensor, the first, second and third regions of the image sensor being non-overlapping.

According to a further feature of an embodiment of the present invention, the internal reflector has a reflectance of at least 20% for at least one wavelength of infrared light incident perpendicular to the major surfaces and a reflectance of less than 10% for a majority of the spectrum of visible light incident perpendicular to the major surfaces.

According to a further feature of an embodiment of the present invention, there is also provided an electronic arrangement associated with the image sensor, the electronic arrangement generating a digital image including a first sub-image corresponding to light sensed in the first region of the image sensor, a second sub-image corresponding to a reflected image of light sensed in the second region of the image sensor and a third sub-image corresponding to a reflected image of light sensed in the third region of the image sensor.

According to a further feature of an embodiment of the present invention, a width of the internal reflector, an effective aperture of the lens and deployment of the image sensor are further configured such that: (i) light from the eye of the user reflected by the internal reflector and reaching the lens after a single reflection from the first major surface followed by a single from the second major surface is incident on a fourth region of the image sensor; and (ii) light from the eye of the user reflected by the internal reflector and reaching the lens after a single reflection from the second major surface followed by a single reflection from the first major surface is incident on a fifth region of the image sensor, the fourth and fifth regions being mutually non-overlapping and being non-overlapping with each of the first, second and third regions.

According to a further feature of an embodiment of the present invention, there is also provided an electronic arrangement associated with the image sensor, the electronic arrangement generating a digital image including a super-position of sub-images corresponding to light sensed in the first, fourth and fifth regions of the image sensor, and sub-images corresponding to a reflected image of light sensed in the second and third regions of the image sensor.

### BRIEF DESCRIPTION OF THE DRAWINGS

The invention is herein described, by way of example only, with reference to the accompanying drawings, wherein:

FIGS. 1A-1C are schematic side views of an arrangement, constructed and operative according to the teachings of an embodiment of the present invention, for forming an image of an eye of a user via a lightguide in facing relation to the user's eye, illustrating, respectively, the light paths from three different areas of an eye motion box (EMB);

FIGS. 2A-2C are schematic illustrations showing the optical arrangement from FIG. 1A, illustrating, respectively, light paths from three closely-spaced points within the EMB around a blind spot of the detector;

FIG. 3A is a test image deployed at the EMB for the purpose of simulations illustrated in the subsequent images;

FIG. 3B is a simulation of the output of an image sensor from the arrangement of FIG. 1A when the test image of FIG. 3A is positioned at the EMB;

FIG. 4A is a copy version of FIG. 3B inverted to correct inversion and mirror reflection of the central lobe of the image;

FIG. 4B is a reconstruction of the source image generated by flipping, swapping and displacing side-lobe sub-images from FIG. 4A;

FIG. 5 is a variant implementation of the arrangement of FIG. 1A;

FIG. 6A is a simulation of the output of an image sensor from the arrangement of FIG. when the test image of FIG. 3A is positioned at the EMB, the image having a central lobe, first-order side lobes and second-order side lobes;

FIG. 6B is a reconstruction of the source image generated by flipping, swapping and displacing the first-order side-lobe sub-images from FIG. 6A, plus global inversion of the image;

FIGS. 7A and 7B are images similar to FIG. 6A illustrating the effect of a displacement of an internal reflector of the arrangement by +1.25 mm, respectively;

FIG. 8 is a further variant implementation of the arrangement of FIG. 1A;

FIG. 9A is a simulation of the output of an image sensor from the arrangement of FIG. 8 when the test image of FIG. 3A is positioned at the EMB, the image having partial redundancy of content;

FIG. 9B is a modified version of FIG. 9A, inverted, and with the side lobes flipped and swapped, so that the image redundancy occurs in adjacent regions to a blind line of the detector;

FIG. 9C is a reconstruction of the source image generated by inward displacement of the side lobes of FIG. 9B and weighted summation of the pixel values in areas of overlap;

FIG. 10 is a graph showing variation of an overlap (correlation) parameter as a function of inward pixel displacement of the side lobes of FIG. 9B, evaluated for three different eye relief distances;

FIG. 11 is a schematic side view of a lightguide illustrating a simplified geometrical analysis to determine the size of an internal reflector and a lens aperture for certain implementations of the present invention; and

FIGS. 12A and 12B are schematic side views of further variant implementations of the arrangement of FIG. 1A illustrating folding of the light path to allow deployment of a camera on a major surface of the lightguide, employing a narrow folding mirror and a wide folding mirror, respectively.

### DESCRIPTION OF THE PREFERRED EMBODIMENTS

The present invention is an arrangement for forming an image of an eye of a user via a lightguide in facing relation to the user's eye.

The principles and operation of arrangements according to the present invention may be better understood with reference to the drawings and the accompanying description.

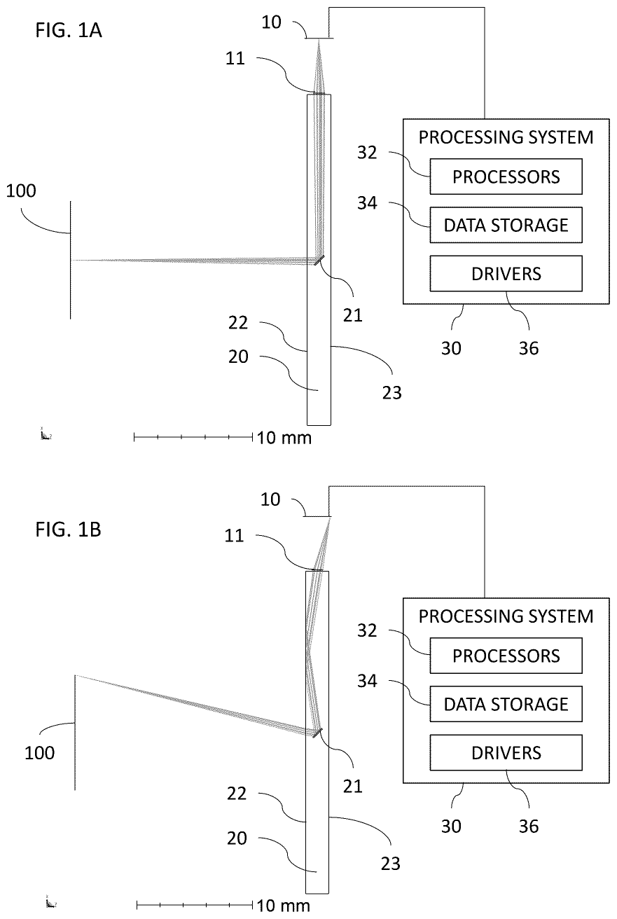

Referring now to the drawings, FIGS. 1A-1C show an arrangement for forming an image of an eye of a user, located within an eye-motion box (EMB) 100, for tracking eye motion. In general terms, the arrangement includes a lightguide 20 formed from transparent material having a first major surface 22 and a second major surface 23, which are planar and mutually parallel. First major surface 22 is deployed in facing relation to the eye of the user, EMB 100, so that the user views a scene through the lightguide. The arrangement also includes an obliquely-angled internal reflector 21 deployed within lightguide 20, an image sensor 10 having a two-dimension array of pixel sensors, and a lens 11 associated with lightguide 20 for focusing light reflected from the eye of the user and reflected by internal reflector 21 onto image sensor 10. A width and angle of internal reflector 21, an effective aperture of lens 11 and deployment of image sensor 10 are such that light reflected from the eye of the user follows various discrete paths to arrive at discrete regions of the image sensor 10. Specifically, a first part of the light reflected from the eye of the user is reflected by internal reflector 21 and reaches lens 11 without reflection from either the first or the second major surface 22 or 23, as illustrated in FIG. 1A, and is incident on a first region of image sensor 10. A second part of the light reflected from the eye of the user is reflected by internal reflector 21 and reaches lens 11 after a single reflection from first major surface 22 and without reflection from the second major surface 23, as illustrated in FIG. 1B, and is incident on a second region of the image sensor 10. A third part of the light reflected from the eye of the user is reflected by internal reflector 21 and reaches lens 11 after a single reflection from second major surface 23 without reflection from first major surface 22, as illustrated in FIG. 1C, and is incident on a third region of image sensor 10. The first, second and third regions of the image sensor are non-overlapping.

Lens 11 is, in this non-limiting example, 40% of a width of the lightguide. Thus, in the example shown, the lightguide width is 2 mm, and the lens width is 0.8 mm. As in any camera, although not shown, the camera should be sealed so as to allow only light propagating through the lens to reach the detector (image sensor 10). Imaging is achieved via lightguide 20 placed approximately 20 mm from EMB 100, which is a typical eye-relief distance for a pair of glasses, although the arrangement may be optimized for any other desired eye-relief distance, depending on the intended application.

Internal reflector (mirror) 21 is preferably oriented so that the chief ray of the center of the EMB propagates parallel to the lightguide major surfaces. In the case of an upright lightguide perpendicular to the line of sight, the internal reflector would thus be at roughly 45 degrees to the major surfaces. In the case of a horizontally-extending reflector in a lightguide deployed with pantoscopic tilt, or in the case of a vertically-extending internal reflector (and imaging at the lateral side of the lightguide) with a face-curve angled deployment, this preferred orientation of the internal reflector may vary from 45 degrees. In the preferred implementation illustrated here, the width of the internal reflector is slightly less than 40% of the maximal width possible inside the lightguide, i.e., spans slightly less than 40% of the lightguide thickness. In the example shown here, the width of the mirror is about 1.1 mm and, when deployed at 45 degrees, the mirror spans 0.78 mm in the thickness direction of the lightguide.

The internal reflector may be implemented as any suitable reflector, including but not limited to, a metallic coating or a multilayer dielectric coating. In a particularly preferred case, the internal reflector is implemented as a multilayer dielectric coating which is configured so to be substantially transparent (reflectivity of less than 10%, and preferably less than 5%) for visible light incident perpendicular to the major surfaces across a majority of the visible spectrum and to be more significantly reflective (reflectivity of at least about 20%, and preferably more than 30%) for at least one wavelength of infrared light incident perpendicular to the major surfaces of the lightguide. Such a reflector is sometimes referred to as a “hot mirror”, being reflective to IR and transparent to visible light.

Most preferably, the mirror should be transparent within the visible spectrum not only to the wearer himself but also for people standing in front of him and observing the glasses at a wider span of angles. Assuming observing angles at +−30 degrees, which correspond to about +−20 deg inside the lightguide material, and assuming that the internal reflector is at about 45 degrees to the lightguide surfaces, which correspond to an angle of incidence of 45+20 degrees, or 25-65 degrees. Ideally, the mirror therefore has low reflectivity (preferably less than 10%, and more preferably less than 5%) across at least a majority of the visible spectrum over a range of angles of incidence from 25-65 degrees, and partial reflectance at a wavelength in the NIR range, for example, around 900 nm, with a reflectivity of at least about 20%, and preferably at least about 30%, over a range of incident angles between about 35 degrees and about 55 degrees.

The present invention preferably operates with active illumination of the user's eye within the EMB at one or more wavelength in the near infrared (NIR) region, to which reflectivity of the internal reflector, sensitivity of the image sensor, and possibly additional components such as a selective passband or notch filter (not shown) deployed before the image sensor, are matched. Details of the illumination arrangement are not shown in the drawings, but may include illumination via LEDs at the periphery of the lightguide or small LEDs embedded inside the lightguide, or illumination via a dedicated lightguide (not shown).

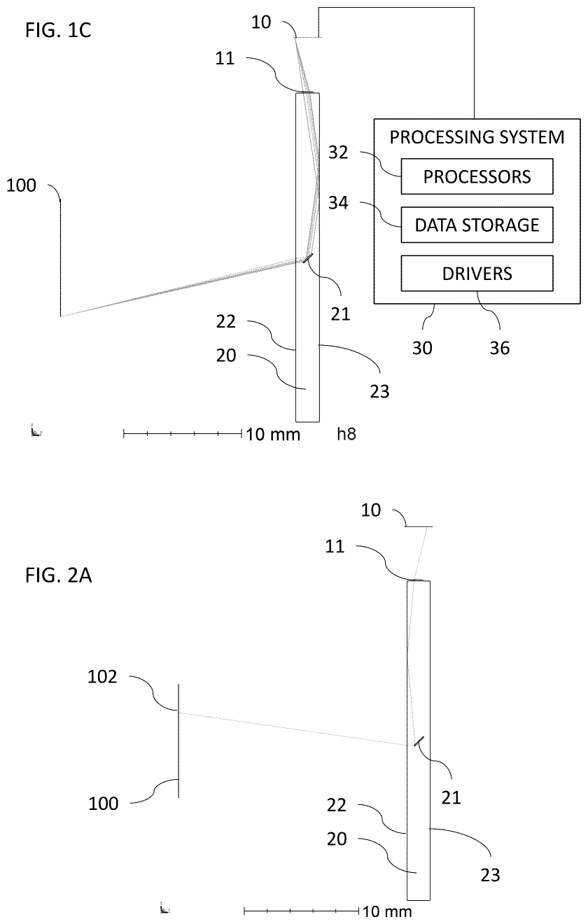

As already mentioned, FIGS. 1A-1C illustrate three different light paths from different regions of the EMB 100 to different regions of image sensor 10. In FIG. 1A, light originating from the center of the EMB hits the folding mirror (internal reflector) 21 and reaches imaging lens 11 without hitting the major surfaces of lightguide 20. FIGS. 1B and 1C illustrate the same structure showing the path of light originating at the edge of the EMB, 5 mm above and below the center, respectively. Light from these regions of the EMB are reflected from one of the major surfaces once and then reach the imaging lens. The narrowness of the folding mirror and the imaging lens ensure that rays propagating along each path form a distinct “lobe” of the image falling on a defined region of the image sensor, and are not mixed. This will be further explained with reference to FIGS. 2A-2C.

FIG. 2A illustrates a ray emerging from a position 102 within the EMB 100 reaches the mirror and into the lens 11 via reflection from the first major surface 22 of the lightguide. In FIG. 2B, we see the beam of light emerging from position 104 and hitting the folding mirror 21. Here it can be seen that all of the rays of the beam do not fall within the aperture of lens 11. At position 106 (FIG. 2C), a ray is shown hitting the edge of mirror 21 and directly propagate towards lens 21. It follows that position 104 is a blind spot of the system, and that the image flips between the two sides of that position.

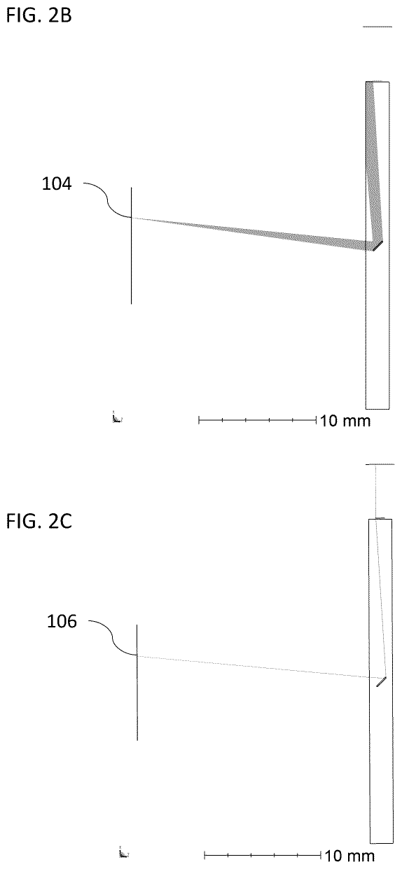

FIG. 3B illustrates the resulting image sensed by detector 10 when the test image of FIG. 3A is provided at EMB 100. As seen, the output image is divided into 3 parts (“lobes”), where the central part corresponds to positions in the EMB the reach the lens directly, without reflection from the major surfaces, but is inverted, like any single-lens imaging system, and is also flipped by the mirror reflection of internal reflector 21. The two side lobes of the image correspond to positions in the EMB from which the light reaches lens 11 after a single reflection at one of the major surfaces, which causes a further mirror-flip of those parts of the image, righting the letters. Notably, although the input test image is continuous, there is a discontinuity between the different lobes of the sensed image.

In many applications, it is desirable to process the detected image to reconstruct an image which faithfully reproduces an approximation of the EMB. Such an image can be used as a direct input into conventional eye-tracking software. To this end, the arrangement of the present invention preferably also includes an electronic arrangement, such as processing system 30 (FIGS. 1A-1C), associated with image sensor 10, that generates a digital image including a first sub-image corresponding to light sensed in the first region of the image sensor, a second sub-image corresponding to a reflected image of light sensed in the second region of the image sensor and a third sub-image corresponding to a reflected image of light sensed in the third region of the image sensor.

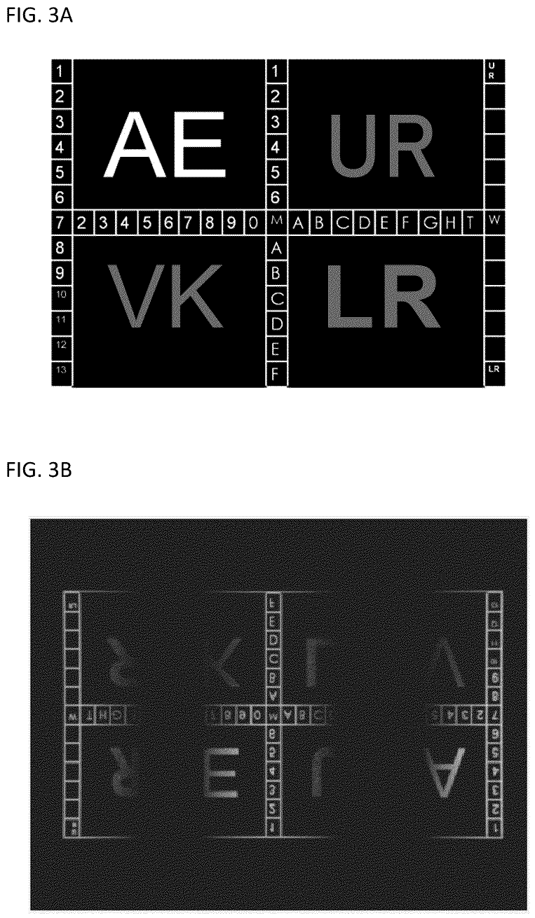

This is illustrated intuitively in FIGS. 4A and 4B. The discussion herein of image manipulation to reconstruct an overall image of the EMB disregards any global manipulations which are applied to the entire image. Thus, FIG. 4A shows the detected image of FIG. 3B after rotation by 180 degrees and flipping horizontally (which is geometrically equivalent to simply flipping FIG. 3B vertically), which renders the central lobe of the image upright. The two side lobes are identified by the two dashed rectangles designated 12 and 13. The corresponding unfolded image is shown in FIG. 4B, where the two side lobes are flipped left-to-right, their positions are swapped, and they are realigned with the center lobe. The resulting image is almost continuous, except for the aforementioned small blind spot (dark vertical line) between the lobes, and a drop-off of intensity towards this blind spot. The blind spot corresponds to position 104 shown in FIG. 2B.

The aforementioned electronic arrangement may be implemented as processing system 30, typically including one or more processors 32, data storage components 34 and drivers 36 or other hardware, software or firmware required to interface with the image sensor and any other components of the system. In many cases, the processing system components may be components which are also used to perform eye-tracking processing and/or to operate a display and/or other components of a system in which the arrangement of the present invention is deployed. In certain cases, it may be possible to implement the electronic arrangement in hardware or firmware of the image sensor, simply by changing the read sequence for columns of the sensor array to correspond to the transformation of FIG. 4B.

It should also be noted that, if a trained neural network is employed for eye tracking processing, it may not be necessary to reconstruct a full image, since the artificial intelligence can be trained to derive eye position directly from raw images such as that of FIG. 4A without the inversion and realignment required to generate a reconstructed image.

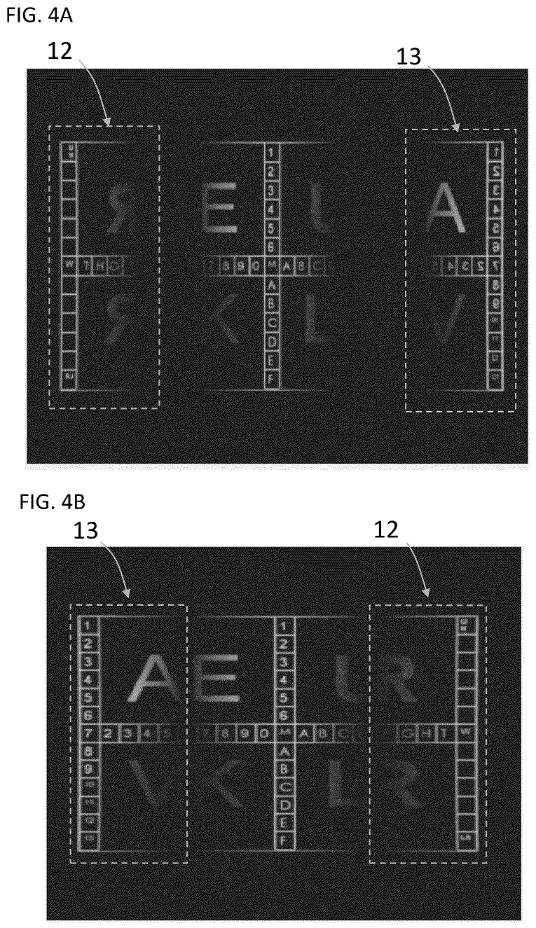

The example illustrated thus derives images formed from three image lobes, where light reaching the image sensor is limited to light paths which are either direct from internal reflector 21 to lens 11, or have undergone exactly one reflection from one of the major surfaces of lightguide 20. However, the invention is not limited to such an implementation, and may in other cases include light paths which have undergone more reflections. As one non-limiting specific example, FIG. 5 illustrates an implementation in which the width of the internal reflector 21, the effective aperture of the lens 11 and deployment of image sensor 10 are such that, in addition to the above paths having zero and one reflections, additional light paths reaching the sensor from the EMB undergo two reflections, one from each of the major surfaces of the lightguide. Thus, as illustrated in FIG. 5, light from a location 108 within EMB 100 is reflected by the internal reflector 21 and reaches lens 11 after a single reflection from first major surface 22 followed by a single from the second major surface 24, and is focused by lens 11 onto a fourth region of image sensor 10. Analogously, light from a location 110 within EMB 100 is reflected by internal reflector 21 and reaches lens 11 after a single reflection from second major surface 23 followed by a single reflection from the first major surface 22, and is focused onto a fifth region of image sensor 10. The light path from location 110 is not illustrated in order to maintain clarity of the drawing, but is analogous to that from location 108. As before, a location 112 in the EMB is reflected by internal reflector 21 so as to reach lens 11 without any reflection from either major surface (forming the first region of the image), and a location 114 (as well as another location between 110 and 112) is reflected so as to undergo a single reflection from only one of the major surfaces before reaching lens 11 (forming the second and third regions of the image). The fourth and fifth regions are mutually non-overlapping and are non-overlapping with each of the first, second and third regions described above. The light paths which are twice-reflected are non-inverted.

The geometrical distinction between the above three-lobe image implementation and this five-lobe image implementation lies primarily in the thickness of the lightguide, the length of the light path from the internal reflector to the lens, and the dimensions of the image sensor. These parameters are preferably chosen so that the image will span the intended EMB dimensions at the expected minimum eye relief distance.

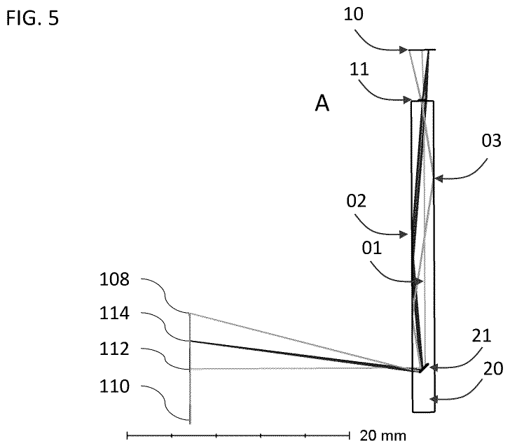

The resulting image is shown in FIG. 6A, where the image is divided into 5 lobes, where the flipped lobes are indicated by two dashed rectangles. The flipped lobes (i.e., those flipped relative to the central lobe) correspond to the light paths which have undergone exactly one reflection at one of the major surfaces of the lightguide, while the other three lobes are all of the same orientation. FIG. 6B illustrates a reconstructed full image (after righting of the central lobe by flipping vertically), where the two flipped lobes are inverted left-to-right, their positions interchanged, and the spaces between different lobes are removed. Such an image can be assembled by the aforementioned electronic arrangement which generates a digital image including a super-position of sub-images corresponding to light sensed in the first, fourth and fifth regions of the image sensor, and sub-images corresponding to a reflected image of light sensed in the second and third regions of the image sensor.

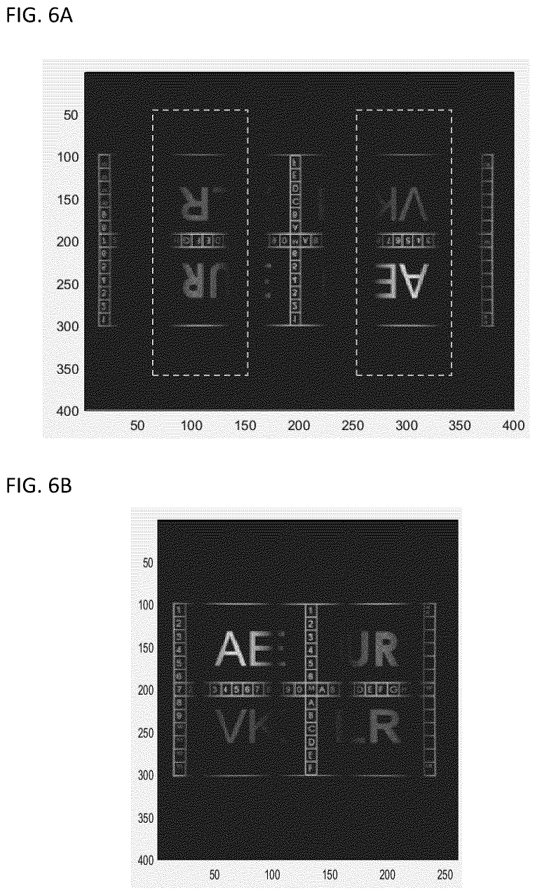

The blind spots of the imaging examples illustrated thus far are not necessarily sufficient to impact the precision of eye tracking, but it may be preferable to employ one or more additional features to further reduce any impact that they might have. A first approach to this issue relates to binocular tracking applications. For most people, the fixation direction for both eyes converge towards a single point in space (or are identical for a distant object). It is therefore possible to employ tracking information from one eye to supplement tracking information which is missing for the second eye in order to estimate an overall gaze direction. To this end, the blind spot of two arrangements deployed for imaging the two eyes of the user may have their blind spots offset relative to each other. This can be readily achieved by implementing two different distances between the folding mirror 21 and the imaging lens 11 and/or by slightly varying the inclination angle of folding mirror 21, thereby generating images with different offsets. This is shown in FIGS. 7A and 7B where two resulting images of shifts of +1.25 mm and −1.25 mm are shown. As seen the blind spot between different images is shifted. For instance, the letters R is cut in FIG. 7A (shift −1.25) and appears in full in FIG. 7B (shift 1.25). The letters U and L appear in FIG. 7A and are cut in FIG. 7B.

In the case of binocular eye tracking, the two images will be of different eyes, and possibly differently aligned relative to the respective EMBs, so the images would not be directly combined. However, it is assumed that any features of one eye which are insufficiently clear from the corresponding image to allow precise eye tracking would be clearly visible for the second eye in the second image, thereby facilitating continuous and high precision tracking of overall eye motion.

In all of the embodiments illustrated in FIGS. 1A-2C and 5, as well as the drawings described further below, the optical elements are illustrated in side view. In the width dimension (into the page as illustrated), internal reflector 21 preferably extends sufficiently to fill the field of view of the camera arrangement defined by lens 11 and image sensor 10, while lens 11 (or some other aperture-defining element located near the lens) defines the optical aperture of the imaging system. For simplicity of construction, internal reflector 21 may be implemented spanning an entire dimension of lightguide 10.

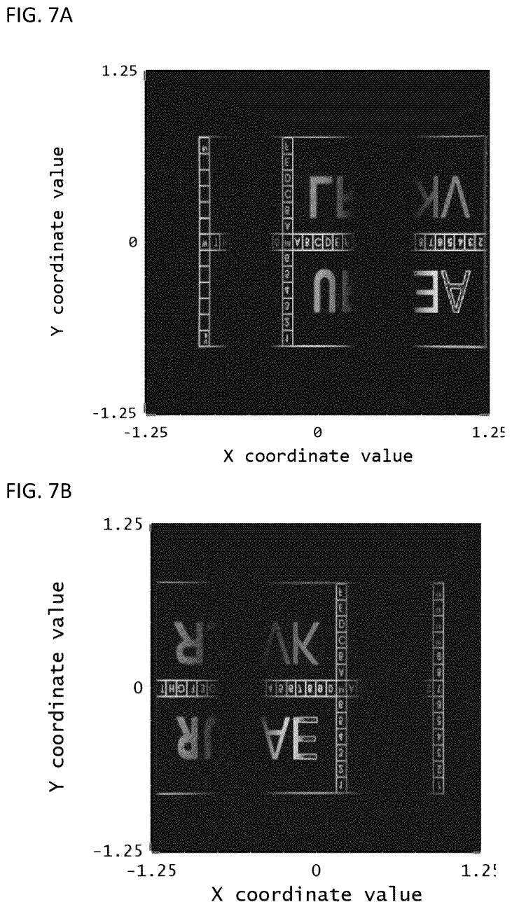

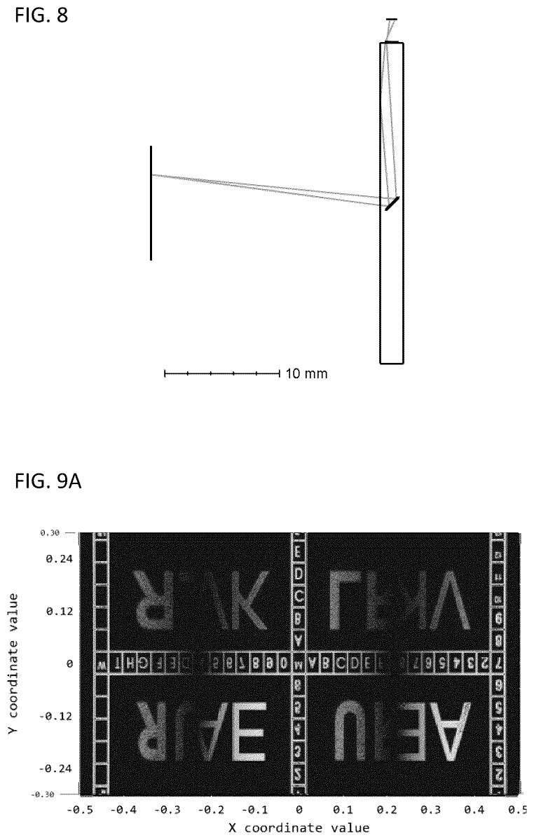

In the examples up to this point, the optical arrangement was such that, for each point in the EMB 100, all rays from that point can reach the imaging camera only via a single type of light path (with either zero, one or two reflections from the major surfaces of the lightguide). However, such an arrangement results in dark spaces on the sensor between the different lobes of the detected image, as seen FIGS. 3B and 6A. Optionally, the size of the aperture defined by the folding mirror 21 and/or the imaging lens 11 can be increased so that light from some areas of the EMB can reach the image detector via more than one light path, but still without any overlap of image regions on the detector. Such a configuration is shown in FIG. 8, where both the mirror and the detector were increased in size. In this example, rays from point 114 within EMB 100 reach the detector along two distinct light paths, so that this area will appear in two different lobes of the image. The resulting image on the detector is shown in FIG. 9A. As can be seen, on the detector, there is no overlap between the different lobes of the image, but some parts of the EMB appear on the detector twice. For instance, part of the letter E appears twice on the detector.

FIG. 9B shows the content of FIG. 9A after a global vertical flip to right the central lobe, and inverting and interchanging the two side lobes of FIG. 9A. It can be seen that the middle lobe and the side lobes share some overlapping content. To generate a faithful reproduction of the image at the EMB, the side lobes and the central lobes should be correctly aligned (e.g., by shifting the side lobes inwards) and their content summed or otherwise combined. After such shifting and weighted summation, the resulting reconstructed image is shown in FIG. 9C. It will be noted that the dark (blind) lines of FIGS. 4B & 6B are here eliminated.

The exact magnitude of shifting of the side lobes required to align them with the central lobe depends on multiple parameters of the system such as the focal length, lightguide thickness, distance between mirror and lens etc. Most of these parameters are properties of the system that do not change and thus could be easily calibrated in advance. However, the shifting also depends slightly on the eye-relief (ER) distance of the system, due to parallax between the edges of the internal reflector. Although the ER does not usually change significantly during use of the glasses, it can vary between different users and even for the same user wearing the glasses on different occasions.

Thus, for some applications, a fixed pre-calibrated offset correction may be used, and may provide acceptable results. Where further optimization is desired, a simple image correlation algorithm can be used to determine the optimal displacement needed. For the purpose of completeness of the disclosure, one algorithm which may be used for this purpose is presented here by way of a non-limiting example. It will be appreciated, however, that various other known algorithms for image correlation may be used for this purpose, as will be clear to a person having ordinary skill in the art. Optionally, the algorithm may be employed to predetermine an optimal correction for various different values of ER distance, and then, when the algorithm is executed for a given user (typically each time the system is switched on, and/or whenever it is repositioned on the face), the output of the algorithm is indicative both of the required offset for combining the image lobes and of the current ER distance.

In the following example, a simulation of the algorithm generates offset corrections of 48 pixels for an ER of 24 mm, 52 pixels for an ER of 20 mm and 58 pixels for an ER of 16 mm. A simple algorithm was used to determine the optimal location where the overlapping-OVL was defined as

$$OVL ( d ) = {\sum }_{i j} {( C {L}_{i , j - d} + S {L}_{i , j} )}^{2}$$

Where i indicates row number, j indicates column number, d indicates the displacement between images, CL is the central lobe, SL is the side lobe and OVL stands for the overlapping or correlation factor to be optimized.

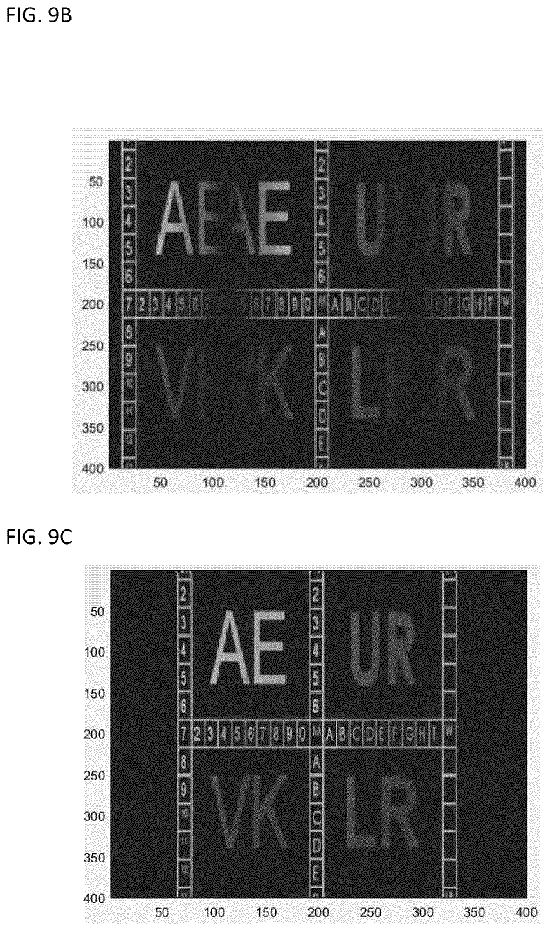

FIG. 10 illustrates parameter OVL calculated as a function of pixel displacement for three different ER distances of the system. As seen, the maximum is easily identified for each simulation, which is then indicative of both the correct displacement and the current ER of the user.

Determining the ER distance for the current user may be useful for operation of the eye tracking function itself, and may also be a useful byproduct, helpful for other aspects of operation of a head-mounted display. For example, knowledge of the ER distance may facilitate derivation of the fixation distance of the user as the ER changes with the distance of focusing.

Various considerations impact the choice of sizes of the internal reflector 21 and the aperture of lens 11. As illustrated above, the use of a relatively narrow reflector and small-aperture lens ensure that each image point appears at only one location in the detector plane, but makes inefficient use of the detector area and suffers from blind spots. By enlarging the width of the internal reflector and the lens aperture, the situation of FIGS. 9A-9C is achieved, where parts of the EMB appear twice at the detector plane, but still avoiding overlap between the different lobes of the image. This makes better use of the detector plane, and allows elimination of the blind spots. However, it is important that the reflector and lens are kept sufficiently small to avoid overlap between the lobes on the detector plane, which would jumble the image content and cause drastic degradation of the output.

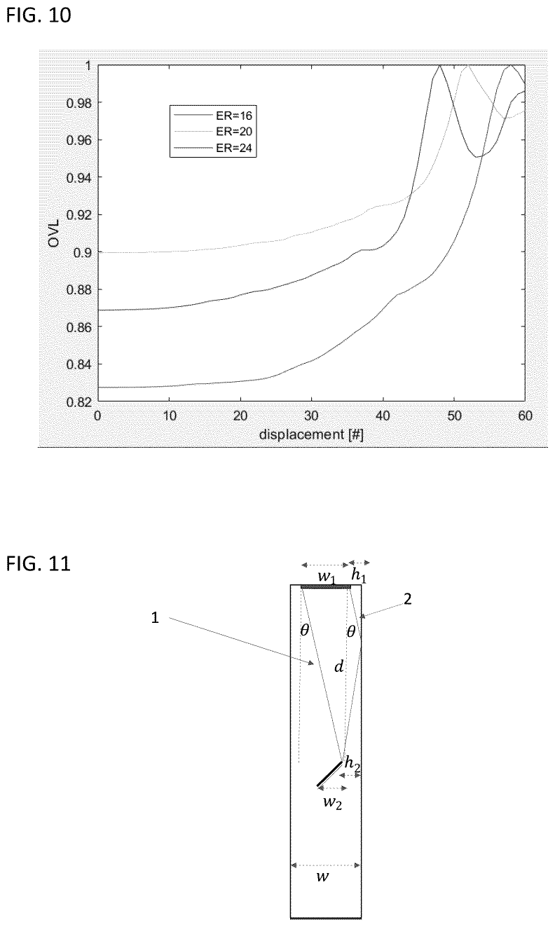

FIG. 11 presents schematically the geometrical considerations to avoid overlap of the image lobes in the detector plane using a simplification of assuming that the imaging distance (eye relief distance) is long, i.e., that the rays from the image are parallel.

The minimal distance between the mirror and the detector is designated d. The width of the detector is designated w₁. The width of the lightguide is w₂ and the width of the lightguide is w. Assuming the detector and the mirror are centered within the lightguide, the distance remaining between the edges mirror and the lightguide major surface can be determined as

$${h}_{2} = \frac{w - {w}_{2}}{2} ,$$

and similarly for the detector aperture

$${h}_{1} = \frac{w - {w}_{1}}{2} .$$

Using our simplification according to which we assume that the camera pupil is set to look to infinity, we want to prevent a single point on the detector from receiving rays from two different locations in the EMB, which would occur if a direct ray and a once-reflected ray from the internal reflector to the lens aperture could enter the lens at the same angle. We therefore look at the propagation of rays exiting from the upper corner of the mirror and hitting the two edges of the aperture so that both different angled incident rays will reach the same point on the detector as seen in FIG. 11.

For the left ray, its angle of propagation is given by

$$tan ( θ ) = \frac{0.5 ( {w}_{1} + {w}_{2} )}{d} .$$

For the right ray 2, we can get a similar equation

$$tan ( θ ) = \frac{{h}_{1} + {h}_{2}}{d} .$$

This is clearly shown in FIG. 11 when we reflect the lightguide around its major surface of hitting of the ray. Assuming as before that the mirror and the pupil of the lens are centered, we can write:

$$\frac{{h}_{1} + {h}_{2}}{d} = \frac{w}{d} - \frac{0.5 ( {w}_{1} + {w}_{2} )}{d} .$$

Comparing the two equations we get

$$\frac{w}{d} - \frac{0.5 ( {w}_{1} + {w}_{2} )}{d} = \frac{0.5 ( {w}_{1} + {w}_{2} )}{d}$$

$$w = {w}_{1} + {w}_{2}$$

In other words, according to this simplification, if the sum of the thickness spanned by internal reflector 21 and the width of the lens aperture add up to less than the thickness of the lightguide, overlap of images on the detector plane should be avoided.

In practice, the imaging distance is not infinite. For shorter imaging distances, the sum of w₁+w₂ can typically be larger than w without causing the image lobes to overlap. Suitable values for the width of the internal reflector and the lens aperture should therefore preferably be determined by numerical and/or empirical methods based on simulations and/or lab experiments, as will be clear to one ordinarily skilled in the art.

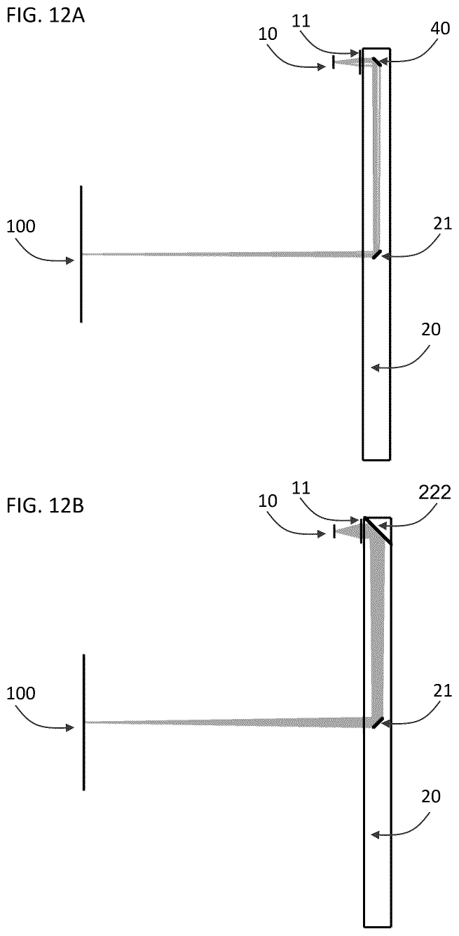

The above examples all depict the camera configuration as being linear or “in-plane”, where the lens and the image detector are deployed to collect image light exiting from an edge of the lightguide. In certain implementations, it may be more convenient to fold the optical axis of the camera using another mirror. Two options for such an implementation are shown in FIGS. 12A and 12B.

In FIG. 12A, a folding mirror 40 is deployed to fold the optical path in front of lens 11 and detector 10. Mirror 40 can be narrow so as to serve as the aperture stop for the lens 11, at least in one dimension, thereby defining the desired optical architecture as analogous to FIG. 1A, or as analogous to FIG. 5. Alternatively, as illustrated in FIG. 12B, a wider mirror 222 may be provided, spanning the width of the lightguide and folding all of the rays propagating within the lightguide towards lens 11. In this case, the lens itself (or an associated aperture stop) serves as the stop. In all other ways, the embodiments of FIGS. 12A and 12B are similar in structure and function to those of FIGS. 1A and 5, described above.

In all of the above examples, the internal reflector 21 preferably passes through a central portion of the lightguide 20 so as to capture images of the eye from near the center of the field of view. Thus, the lightguide is typically supported by a support structure (such as a glasses frame supported on the nose and ears of the user, or a head-mounted visor) which supports a lightguide in facing relation to each eye of the user, and the internal reflector preferably passes within about 20 degrees, and more preferably within about 10 degrees, of the center of the user's field of view, corresponding to the axis along which the user views the outside world when looking straight ahead. This central positioning provides a front view of the eye, which simplifies eye tracking processing and/or enhances eye tracking precision.

The arrangement of the present invention is preferably combined with a display, which delivers a visible image to the user's eye, either via the same lightguide as the arrangement of the present invention or via another parallel lightguide, employing reflective coupling-out or diffractive coupling-out of the image, all as is known in the art.

It will be appreciated that the above descriptions are intended only to serve as examples, and that many other embodiments are possible within the scope of the present invention as defined in the appended claims.

What is claimed is:

1. An arrangement for forming an image of an eye of a user for tracking eye motion, the arrangement comprising:
(a) a lightguide formed from transparent material having a first major surface and a second major surface, said first and second major surfaces being planar and mutually parallel, said first major surface being deployed in facing relation to the eye of the user so that the user views a scene through said lightguide;
(b) an obliquely-angled internal reflector deployed within said lightguide;
(c) an image sensor comprising a two-dimension array of pixel sensors; and
(d) a lens associated with said lightguide for focusing light reflected from the eye of the user and reflected by said internal reflector onto said image sensor,
wherein a width of said internal reflector, an effective aperture of said lens and deployment of said image sensor are such that:
(i) light from the eye of the user reflected by said internal reflector and reaching said lens without reflection from either said first or said second major surface is incident on a first region of said image sensor;
(ii) light from the eye of the user reflected by said internal reflector and reaching said lens after a single reflection from said first major surface and without reflection from said second major surface is incident on a second region of said image sensor; and
(iii) light from the eye of the user reflected by said internal reflector and reaching said lens after a single reflection from said second major surface and without reflection from said first major surface is incident on a third region of said image sensor,
said first, second and third regions of said image sensor being non-overlapping.

2. The arrangement of claim 1, wherein said internal reflector has a reflectance of at least 20% for at least one wavelength of infrared light incident perpendicular to said major surfaces and a reflectance of less than 10% for a majority of the spectrum of visible light incident perpendicular to said major surfaces.

3. The arrangement of claim 1, further comprising an electronic arrangement associated with said image sensor, said electronic arrangement generating a digital image including a first sub-image corresponding to light sensed in said first region of said image sensor, a second sub-image corresponding to a reflected image of light sensed in said second region of said image sensor and a third sub-image corresponding to a reflected image of light sensed in said third region of said image sensor.

4. The arrangement of claim 1, wherein a width of said internal reflector, an effective aperture of said lens and deployment of said image sensor are further configured such that:
(i) light from the eye of the user reflected by said internal reflector and reaching said lens after a single reflection from said first major surface followed by a single from said second major surface is incident on a fourth region of said image sensor; and
(ii) light from the eye of the user reflected by said internal reflector and reaching said lens after a single reflection from said second major surface followed by a single reflection from said first major surface is incident on a fifth region of said image sensor,
said fourth and fifth regions being mutually non-overlapping and being non-overlapping with each of said first, second and third regions.

5. The arrangement of claim 4, further comprising an electronic arrangement associated with said image sensor, said electronic arrangement generating a digital image including a super-position of sub-images corresponding to light sensed in said first, fourth and fifth regions of said image sensor, and sub-images corresponding to a reflected image of light sensed in said second and third regions of said image sensor.
